(19) United States
(12) Patent Application Publication
(10) Pub. No.: US 2008/0147183 A1
(43) Pub. Date: Jun. 19, 2008

(54) Endovalve

(76) Inventor: Mikolaj Styrc, Kopstal (LU)

Correspondence Address: WENDEROTH, LIND & PONACK, L.L.P., 2033 K STREET N. W., SUITE 800, WASHINGTON, DC, 20006-1021, US

(21) Appl. No.: 12/000,422

(22) Filed: Dec. 12, 2007

(30) Foreign Application Priority Data
Dec. 14, 2006 (FR) 06 10909

(51) Int. Cl. A61F 2/24

(52) U.S. Cl. 623/2.12

(57) ABSTRACT

The endovalve comprises an endoprosthesis deployable between a contracted state and an expanded state. The endoprosthesis defines an internal duct. The endovalve includes a valve placed in the internal duct and movable between a closed configuration shutting the duct and a release configuration leaving the duct open. The endovalve has connection means between the valve and the endoprosthesis. The connection means comprise at least one intermediate connection member connected to the endoprosthesis and including at least one region that is movable relative to the endoprosthesis. The valve is fastened to the movable region.

The present invention relates to an endovalve of the type comprising:
an endoprosthesis deployable between a contracted state and an expanded state, the endoprosthesis defining an internal duct;
a valve placed in the internal duct and movable between a closed configuration shutting the internal duct and a release configuration leaving the internal duct open; and
connection means between the valve and the endoprosthesis.

### BACKGROUND OF THE INVENTION

Such endovalves are intended in particular to replace defective native valves in the human heart.

An endovalve of the above-specified type is known that is implanted as a replacement for a native valve by the endoluminal approach. To do this, the endovalve is taken to its point of implantation while in a contracted state, e.g. in a catheter. The endovalve is then deployed in register with the native valve that needs to be replaced, and becomes pressed against the wall defining the native valve.

In order to ensure that the valve carried by the endovalve operates properly, it is necessary for it to be fastened securely to the endoprosthesis that constitutes the structure bearing against the wall.

Consequently, it is known to stitch the valve in the endoprosthesis so as to fasten it securely to the filaments constituting the deployable trellis of the endoprosthesis. The stitching is performed outside the human body, while the endoprosthesis is in its expanded state, in a uniform configuration.

Nevertheless, after the endoprosthesis has been implanted it has a shape that depends on the morphology of the patient in which it is implanted. Thus, if the configuration of the native valve does not present a cross-section that is cylindrical, then the endoprosthesis in its expanded state will occupy a section that might be elliptical, or even triangular. Such shapes that are not radially uniform create stresses on the valve fastened to the trellis, thereby tending to damage the valve or to interfere with its operation.

### OBJECTS AND SUMMARY OF THE INVENTION

An object of the invention is thus to provide an endovalve capable of being implanted in satisfactory manner in patients presenting a variety of morphologies, without harming the reliability of its operation.

To this end, the invention provides an endovalve of the above-specified type, wherein the connection means comprise at least one intermediate connection member connected to the endoprosthesis and including at least one region that is movable relative to the endoprosthesis, the valve being fastened to the or to at least one movable region.

The endovalve of the invention may include one or more of the following characteristics taken in isolation or in any technically feasible combination:
the endoprosthesis has a side wall defining at least one transverse opening into the internal duct and to the outside of the endoprosthesis through the side wall, the assembly formed by the intermediate connection member and the valve being engaged in the transverse opening;
the assembly formed by the valve and the intermediate connection member is movable locally relative to the side wall over a stroke that is defined by the transverse opening;
the intermediate connection member has an outside region placed outside the endoprosthesis and an intermediate region connecting the or each movable region via a transverse opening of the endoprosthesis;
the intermediate connection member is movable locally along its entire length relative to the endoprosthesis;
the intermediate connection member is placed outside the endoprosthesis, the valve being fastened to the intermediate connection member via at least one transverse opening formed in the endoprosthesis along a line of fastening;
the intermediate connection member is of a shape that is substantially complementary to the shape of the line of fastening;
the intermediate connection member is a filamentary tie;
the intermediate connection member is a ribbon; and
the intermediate connection member is a tubular sleeve placed around the endoprosthesis.

### BRIEF DESCRIPTION OF THE DRAWINGS

The invention can be better understood on reading the following description given purely by way of example and made with reference to the accompanying drawings, in which:

FIG. 1 is a face view of a first endovalve of the invention;

FIG. 2 is a section view on horizontal cross-section plane II showing the endovalve of FIG. 1;

FIG. 3 is a view analogous to FIG. 1 showing a second endovalve of the invention;

FIG. 4 is a view analogous to FIG. 2 showing the second endovalve of the invention;

FIG. 5 shows two examples of intermediate fastener ribbons for the second valve of the invention;

FIG. 6 is a view analogous to FIG. 1 showing a third endovalve of the invention; and

FIG. 7 is a view analogous to FIG. 2 showing a third endovalve of the invention.

### MORE DETAILED DESCRIPTION

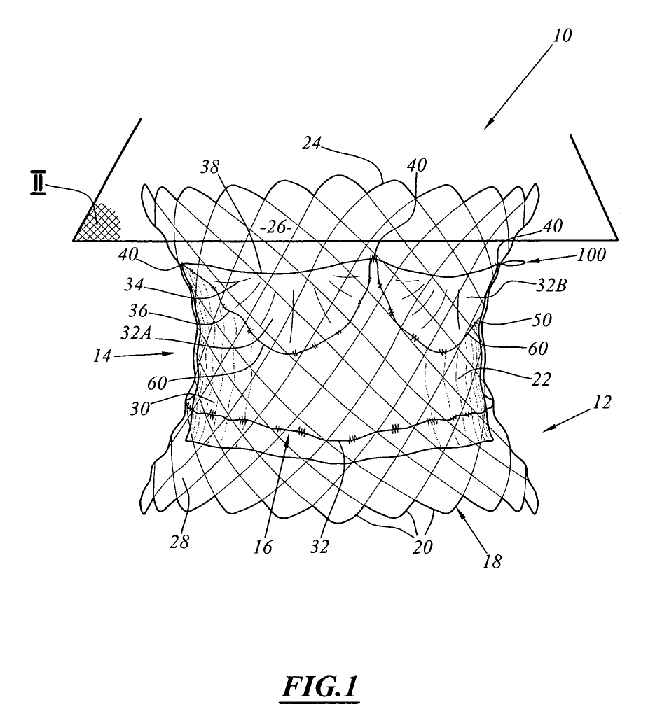

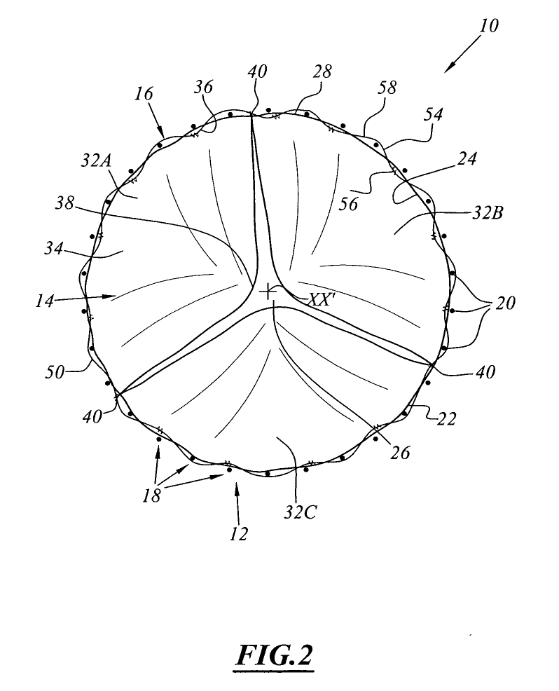

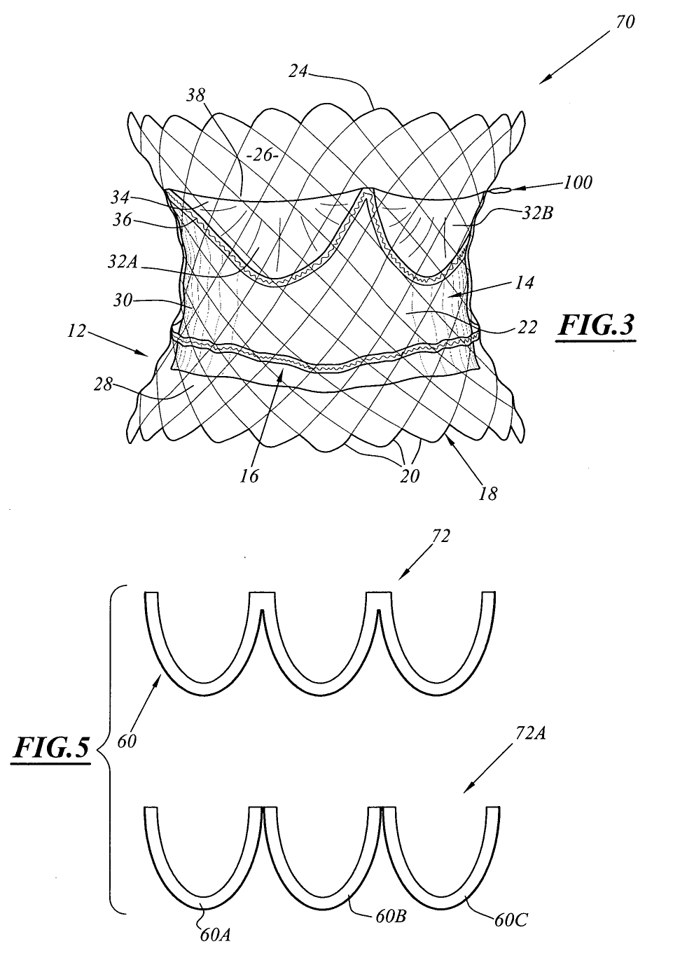

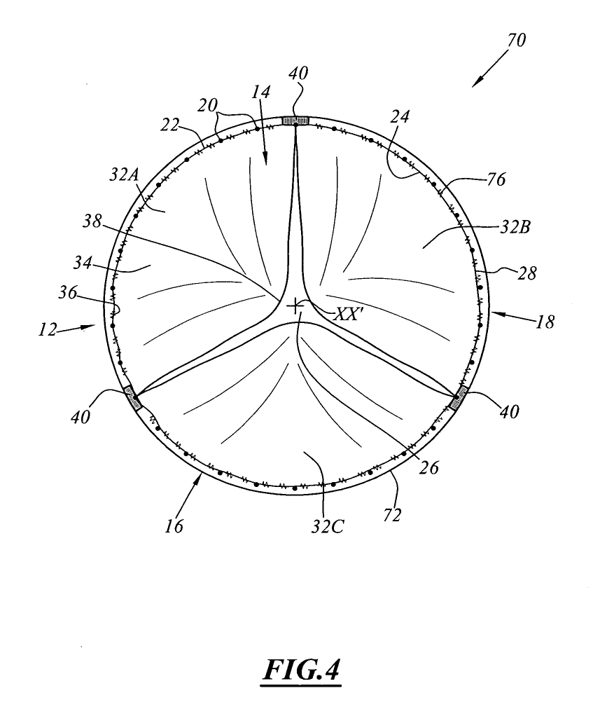

The first endovalve 10 of the invention, shown in FIGS. 1 and 2, is for replacing a defective native heart valve via the endoluminal approach.

The endovalve 10 comprises a tubular endoprosthesis 12, a valve 14 placed in the endoprosthesis 12, and intermediate connection means 16 between the endoprosthesis 12 and the valve 14.

The tubular endoprosthesis 12 is made up of a frame constituted by a tubular trellis 18 that possesses spring properties. The trellis 18 is obtained by braiding at least one filament of stainless steel, of a shape memory alloy, or of a polymer. In a variant, the trellis is obtained by using a laser to cut it out from a tube.

The trellis 18 comprises a plurality of filamentary segments 20 that cross over one another and define lozenge-shaped transverse openings 22.

The trellis 18 defines an inside surface 24 defining an internal duct 26 through which blood flows, and an outside surface 28 for pressing against the wall of a blood vessel, in register with the defective native valve.

The transverse openings 22 open out into the inside surface 24 and into the outside surface 28.

The metal trellis 18 of the endoprosthesis 12 is deployable between a contracted state in which it presents a small diameter and an expanded state, constituting its rest state, in which it presents a large diameter.

In the example shown in FIG. 1, the trellis 18 is spontaneously deployable between its contracted state and its expanded state. In a variant, a balloon is used for deploying it.

The valve 14 is made for example on the basis of a native valve from an animal such as a pig. In a variant, it is made on the basis of natural or synthetic fabric.

It comprises a tubular base 30 extended upwards by three duct-closing flaps 32A, 32B, and 32C.

The base 30 presents an outside surface that is substantially complementary to the inside surface 24 of the endoprosthesis 12, against which it is applied.

In the example shown in FIG. 2, the flaps 32A, 32B, and 32C are made integrally with the base 30 which they extend upwards.

The flaps 32A, 32B, and 32C are distributed around the axis X-X′ of the duct 26. Each flap 32A, 32B, and 32C thus occupies an angular sector of about 120° about the axis X-X′.

Each flap 32A, 32B, and 32C is in the form of a pouch-shaped membrane 34 extending facing the portion of the inside surface 24. Each flap 32A, 32B, and 32C is defined outwards and downwards by a substantially U-shaped fastener line 36, and upwards by a free edge 38 that is movable in the duct 26.

The fastener line 36 extends substantially over the inside surface 24 of the endoprosthesis 12 to two top ends 40 forming the side ends of the free edge 38. Each top end 40 of a flap 32A, 32B, and 32C is common between two adjacent flaps 32A, 32B, and 32C.

As explained below, the fastener lines 36 are stitched exclusively onto intermediate connection means 16 and not onto the trellis 18 of the endoprosthesis 12.

The free edge 38 extends substantially horizontally within the internal duct 26 between the top ends 40.

The free edges 38 and the pouches 34 can be moved under the effect of the pressure of blood in the internal duct 26, between a release configuration for opening the duct 26 (not shown) and a closed configuration for shutting the duct 26, as shown in FIG. 2.

In the release configuration, the pouches 34 are pressed against the inside surface 24 of the endoprosthesis 12. The free edges 38 then extend the surface 24 and are spaced apart from one another.

The distance between each free edge 38 and the axis X-X′ is then at a maximum. The area occupied by the flaps 32A, 32B, and 32C in the duct 26 is then at a minimum to allow blood to pass through.

In the closed configuration, the pouches 34 are deployed inside the duct 26. The free edges 38 are situated close to the axis X-X′. The free edge 38 of each flap 32A, 32B, and 32C is pressed over substantially its entire length against the free edges 38 of the adjacent flaps 32B, 32C, and 32A.

In this configuration, the pouches 34 occupy a maximum area within the duct 26, and blood is substantially prevented from passing along the duct 26.

According to the invention, the connection means 16 between the valve 14 and the endoprosthesis 12 are formed by intermediate connection members connected to the endoprosthesis 12, but including at least one region that is displaceable relative to the endoprosthesis 12.

In the first endovalve 10 of the invention, the connection means 16 are formed by a top connection filament 50 for fastening to the flaps 32A, 32B, and 32C, and by a bottom connection filament 52 for fastening to the base 30.

The filaments 50, 52 are interlaced with the trellis 18 of the endoprosthesis 12 between the inside surface 24 and the outside surface 28. The filaments 50, 52 thus pass successively inside and outside the filamentary segments 20 constituting the trellis 18 by passing through the openings 22.

The filaments 50, 52 are thus connected to the endoprosthesis in that they can move together with the endoprosthesis 12 when the endoprosthesis 12 is moved over a long distance.

Nevertheless, the filaments 50, 52 are locally movable over their entire length relative to the endoprosthesis 12, over a stroke that is defined by the size of each opening 22 in which the filament 50, 52 is engaged.

Each filament 50, 52 defines a plurality of outside regions 54 situated on the outside of the endoprosthesis 12, a plurality of inside fastener regions 56 placed inside the internal duct 26, and a plurality of intermediate regions 58, each intermediate region 58 connecting a region 54 to a region 56 via a transverse hole 22.

The top connection filament 50 presents three U-shaped spans 60 extending respectively facing the fastener lines 36 of each flap 32A, 32B, and 32C.

The flaps 32A, 32B, and 32C are stitched to the inside fastener regions 56. The flaps 32A, 32B, and 32C and the filament 50 thus form a single assembly engaged in a plurality of transverse openings 22.

Similarly, the bottom connection filament 52 extends along the bottom edge of the base 30 around a circumference of the inside surface 24 about the axis X-X′. The base 30 of the valve 14 is stitched to the inside fastener regions 56 of the bottom connection filament 52, along said circumference.

The valve 14 is thus fastened overall relative to the tubular endoprosthesis 12 so as to be moved together with the endoprosthesis 12 by co-operation between the trellis 18 and the assembly constituted by the valve 14 together with the intermediate connection filaments 50, 52. The assembly formed by the valve 14 and the filaments 50, 52 is also movable locally relative to the endoprosthesis 12 in the openings 22, following a stroke that is defined by the size of each opening 22.

The operation of the first endovalve 10 of the invention is described below.

Initially, during fabrication of the endovalve 10, the filaments 50, 52 are engaged in the trellis 18 of the endoprosthesis 12 to form three respective U-shaped spans 60 around the axis X-X′, and to extend along a bottom circumference of the trellis 18. The valve 14 is then inserted into the duct 26 and is stitched to the inside fastener regions 56 of the filaments 50, 52.

The endoprosthesis 12 is then retracted into its contracted state and is maintained in this state by release means (not shown) for releasing the endovalve 10 in the organism, and constituted for example by an outer sheath or by a filamentary release system as described in application FR-A-2 863 160 in the name of the Applicant.

Thereafter, the release means transporting the endovalve 10 are inserted into the patient and taken to the implantation site, e.g. using an endoluminal approach.

When the endovalve 10 is situated in register with its implantation site, in the vicinity of the native valve, the release means are operated to cause the endovalve 10 to pass from its contracted state to its expanded state. The outside surface 28 of the endoprosthesis 12 is then pressed against the wall of a blood-flow duct. The endoprosthesis 12 then presents a cross-section that is substantially complementary to the section of the duct in which it is applied.

Nevertheless, even if the trellis 18 does not deploy uniformly around the axis X-X′, i.e. if some of the transverse openings 22 of the trellis 18 are more deformed than others, any stresses on the valve 14 are released by local movements of the filaments 50, 52 and of the valve 14 relative to the trellis 18 in the openings 22. The valve 14 thus occupies a relaxed state, thereby increasing its reliability in operation over time.

Furthermore, since the valve 14 is fastened solely by means of the intermediate connection filaments 50, 52, without being fastened directly to the filamentary segments 20 constituting the trellis 18, the deployment of the trellis 18 on the endoprosthesis 12 passing from the contracted state to the expanded state is not impeded by the presence of the valve 14, thereby making it easier to release the endoprosthesis 12.

In a variant, the filaments 50, 52 are placed around the outside surface 28 of the endoprosthesis 12. The filaments 50, 52 are fastened to the trellis 18 at certain points, e.g. at least three points around the periphery.

The valve 14 is fastened to the filaments 50, 52 via the transverse openings 22 in free regions of the filaments 50, 52 that are situated between the points where they are fastened to the trellis 18.

In another variant, the valve 14 has a tubular wall surrounding and carrying the flaps 32A, 32B, and 32C. The tubular wall is pressed against the inside surface 24.

Under such circumstances, the top filament 50 presents an outline that is substantially circular, like that of the bottom filament 52. The tubular wall of the valve 14 is then fastened to the filaments 50, 52.

The second endovalve 70 of the invention differs from the first endovalve 10 in that the connection means 16 comprise intermediate members in the form of fabric ribbons 72, 74 of shapes substantially complementary to the shapes of the lines of stitching 36 of the valve 14. Thus, the top fastener ribbon 72 presents three U-shaped spans 60 that are stitched to one another via their top ends. The bottom ribbon 74 presents a ring shape.

By way of example, the ribbons 72, 74 are made using a biologically compatible fabric that is woven or knitted, such as Dacron®.

The ribbons 72, 74 are pressed against the outside surface 28 of the endoprosthesis 12, outside the internal duct 26 and they are connected to the valve 14 by stitches 76 made through the openings 22.

The operation of this second endovalve 70 is substantially analogous to that of the first endovalve 10.

In the variant shown in part in FIG. 5, the fastener ribbon 72A comprises three independent spans 60A, 60B, and 60C that are not connected to one another. Each span 60A, 60B, and 60C is fastened respectively to the fastener line 36 of a respective flap 32A, 32B, and 32C.

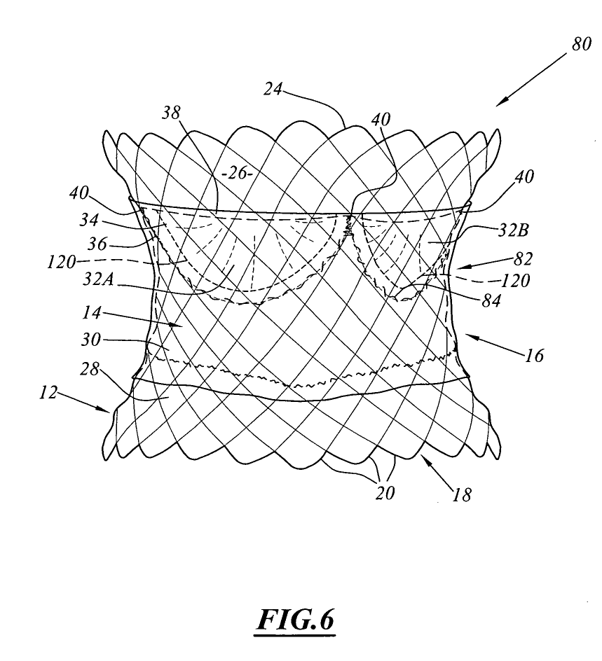

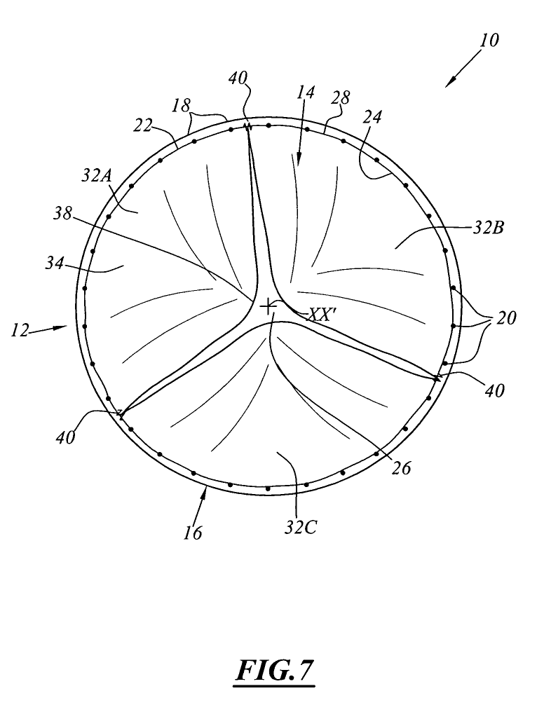

The third endovalve 80 of the invention, shown in FIGS. 6 and 7 differs from the second endovalve 70 in that the intermediate connection member of the connection means 16 is formed by a single sleeve 82 of fabric engaged around the outside surface 28 of the endoprosthesis 12.

The sleeve 82 presents a height that is substantially equal to the height of the valve 14. Stitches 84 secure the flaps 32A, 32B, and 32C and the base 30 securely to the sleeve 82 via the transverse openings 22.

The operation of this third endovalve 80 of the invention is analogous to that of the first endovalve 10 or of the second endovalve 70.

In a variant shown in dashed lines in FIG. 6, the sleeve 82 defines three U-shaped notches 120, each extending in register with a respective flap 32A, 32B, and 32C over the fastener lines 36 of the flaps 32A, 32B, and 32C. The notches 120 open out upwardly into the top edge of the sleeve 82, so as to allow blood to move the flaps 32A, 32B, and 32C from their release configuration pressed against the inside surface 24 towards their closed configuration shutting the duct 26.

In a variant shown in dashed lines in FIG. 1, the top filament 50 is provided with a loop 100 projecting outwards from the surface 28 so as to enable the endovalve 10 to be recovered by pulling on the filament 50 as described in French patent application 06/02932 in the name of the Applicant.

What is claimed is:

1. An endovalve of the type comprising:
an endoprosthesis deployable between a contracted state and an expanded state, the endoprosthesis defining an internal duct;
a valve placed in the internal duct and movable between a closed configuration shutting the internal duct and a release configuration leaving the internal duct open; and
connection means between the valve and the endoprosthesis;
wherein the connection means comprise at least one intermediate connection member connected to the endoprosthesis and including at least one region that is movable relative to the endoprosthesis, the valve being fastened to the or to at least one movable region.

2. An endovalve according to claim 1, wherein the endoprosthesis has a side wall defining at least one transverse opening, opening into the internal duct and to the outside of the endoprosthesis through the side wall, the assembly formed by the intermediate connection member and the valve being engaged in the transverse opening.

3. An endovalve according to claim 2, wherein the assembly formed by the valve and the intermediate connection member is movable locally relative to the side wall over a stroke that is defined by the transverse opening.

4. An endovalve according to claim 2, wherein the intermediate connection member has an outside region placed outside the endoprosthesis and an intermediate region connecting the or each movable region via a transverse opening of the endoprosthesis.

5. An endovalve according to claim 1, wherein the intermediate connection member is movable locally along its entire length relative to the endoprosthesis.

6. An endovalve according to claim 1, wherein the intermediate connection member is placed outside the endoprosthesis, the valve being fastened to the intermediate connection member via at least one transverse opening formed in the endoprosthesis along a line of fastening.

7. An endovalve according to claim 6, wherein the intermediate connection member is of a shape that is substantially complementary to the shape of the line of fastening.

8. An endovalve according to claim 1, wherein the intermediate connection member is a filamentary tie.

9. An endovalve according to claim 1, wherein the intermediate connection member is a ribbon.

10. An endovalve according to claim 1, wherein the intermediate connection member is a tubular sleeve placed around the endoprosthesis.
